(19) United States
(12) Patent Application Publication
(10) Pub. No.: US 2026/0151247 A1
(43) Pub. Date: Jun. 4, 2026

(54) METHOD FOR CONTROLLING ANKLE INVERSION AND EVERSION USING AN ADJUSTABLE ANKLE BRACE

(71) Applicant: BRYAN E. KILBEY, DeFuniak Springs, FL (US)

(72) Inventor: BRYAN E. KILBEY, DeFuniak Springs, FL (US)

(21) Appl. No.: 19/457,391

(22) Filed: Jan. 23, 2026

Related U.S. Application Data
(63) Continuation of application No. 16/666,985, filed on Oct. 29, 2019, now abandoned.

(51) Int. Cl. A61F 5/01

(52) U.S. Cl. CPC A61F 5/0111

(57) ABSTRACT

A method of controlling ankle inversion and eversion using an ankle-brace that incorporates an anti-rotation feature. The brace includes a conventional unified foot and ankle enclosure. An anterior break in the enclosure is secured by a closure device-preferably one or more laces. A tongue preferably spans the break beneath the closure device. One or more lateral straps are provided. Each of these straps is configured to be wrapped around the anterior side of the enclosure, wrapped under the enclosure, and attached to the side of the enclosure. The outward facing surface of the enclosure includes a hook-compatible covering, at least across the region of the sole. Each lateral strap includes a hook panel positioned to engage the hook-compatible covering on the sole. This engagement prevents slippage between the lateral strap and the unified foot and ankle enclosure.

### CROSS-REFERENCES TO RELATED APPLICATIONS

This non-provisional patent application is a continuation of U.S. patent application Ser. No. 16/666,985. The parent application listed the same inventor. It was filed on Oct. 29, 2019.

### STATEMENT REGARDING FEDERALLY SPONSORED RESEARCH OR DEVELOPMENT

Not Applicable

### MICROFICHE APPENDIX

Not Applicable

### BACKGROUND OF THE INVENTION

### 1 Field of the Invention

This invention relates to the field of medical products. More specifically, the invention comprises a method of limiting ankle inversion and eversion using an adjustable ankle brace.

### 2. Description of the Related Art

Orthotic braces are available for many parts of the body. Because the human ankle is particular susceptible to injury, many different types of ankle braces have been developed. A very common injury involves “rolling” the ankle. Rolling injuries typically involve excess pronation (eversion) or excess supination (inversion). A brace may be used following such an injury. The brace limits inversion and/or eversion during the healing process. The limitation on motion is important to prevent aggravation and re-injury.

Many prior art ankle braces include a “FIG. 8” wrapping in which a pair of straps are wrapped over the anterior ankle in opposite directions. The straps are then wrapped under the sole of the foot and secured back to the sides of the ankle brace. These straps provide some limitation on the unwanted inversion and eversion motions. However, the straps often slip along the outward-facing surfaces of the ankle brace. This slippage allows inversion and/or eversion despite the presence of the brace. The present invention eliminates this deficiency.

### BRIEF SUMMARY OF THE PRESENT INVENTION

The present invention comprises a method of controlling ankle inversion and eversion using an ankle-brace that incorporates an anti-rotation feature. The brace includes a conventional unified foot and ankle enclosure. An anterior break in the enclosure is secured by a closure device—preferably one or more laces. A tongue preferably spans the break beneath the closure device. One or more lateral straps are provided. Each of these straps is configured to be wrapped around the anterior side of the enclosure, wrapped under the enclosure, and attached to the side of the enclosure.

The outward facing surface of the enclosure includes a hook-compatible covering, at least across the region of the sole. Each lateral strap includes a hook panel positioned to engage the hook-compatible covering on the sole. This engagement prevents slippage between the lateral strap and the unified foot and ankle enclosure.

### BRIEF DESCRIPTION OF THE SEVERAL VIEWS OF THE DRAWINGS

FIG. 1 is a perspective view, showing the inventive ankle brace.

FIG. 2 is a perspective view, showing the inventive ankle brace from a different vantage point.

FIG. 3 is a perspective view, showing the inventive ankle brace installed on a user's foot and ankle.

FIG. 4 is a perspective view, showing the configuration of FIG. 3 with the upper and lower laces tightened.

FIG. 5 is a detailed perspective view, showing the upper rear portion of the ankle brace.

FIG. 6 is a perspective view, showing the placement of the first side lateral strap.

FIG. 7 is a perspective view, showing the placement of the first side lateral strap.

FIG. 8 is a perspective view, showing the placement of the second side lateral strap.

FIG. 9 is a perspective view, showing the placement of the second side lateral strap.

FIG. 10 is a perspective view, showing an anti-rotation feature of the present invention.

FIG. 11 is a perspective view, showing an anti-rotation feature of the present invention.

FIG. 12 is a bottom view, showing the placement of the anti-rotation features of the present invention.

FIG. 13 is a rear elevation view, showing the operation of the anti-rotation features.

FIG. 14 is a perspective view, showing the addition of a superior securing strap.

REFERENCE NUMERALS IN THE DRAWINGS

| | |
|---|---|
| 10 | ankle brace |
| 12 | foot enclosure |
| 14 | ankle enclosure |
| 16 | heel opening |
| 18 | edge band |
| 20 | edge band |
| 22 | first side reinforcement panel |
| 23 | second side reinforcement panel |
| 24 | upper lace |
| 26 | lower lace |
| 28 | upper tab |
| 30 | lower tab |
| 32 | first side lower eye |
| 34 | second side lower eye |
| 36 | loop |
| 38 | anterior foot opening |
| 40 | second side upper eye |
| 42 | first side upper eye |
| 44 | tongue |
| 46 | adjustment break |
| 48 | internal loop |
| 50 | ankle |
| 52 | foot |
| 54 | inferior tongue edge |
| 56 | superior perimeter |
| 58 | first side lateral strap |
| 59 | superior securing strap |
| 60 | second side lateral strap |
| 62 | pull tab |
| 64 | hook panel |
| 66 | loop panel |
| 68 | distal end |
| 70 | distal end |
| 72 | pull tab |
| 73 | hook panel |
| 74 | hook-compatible panel |
| 76 | hook-compatible panel |
| 78 | inferior region |
| 80 | hook panel |
| 82 | heel |
| 84 | hook panel |
| 92 | interior surface |

### DETAILED DESCRIPTION OF THE INVENTION

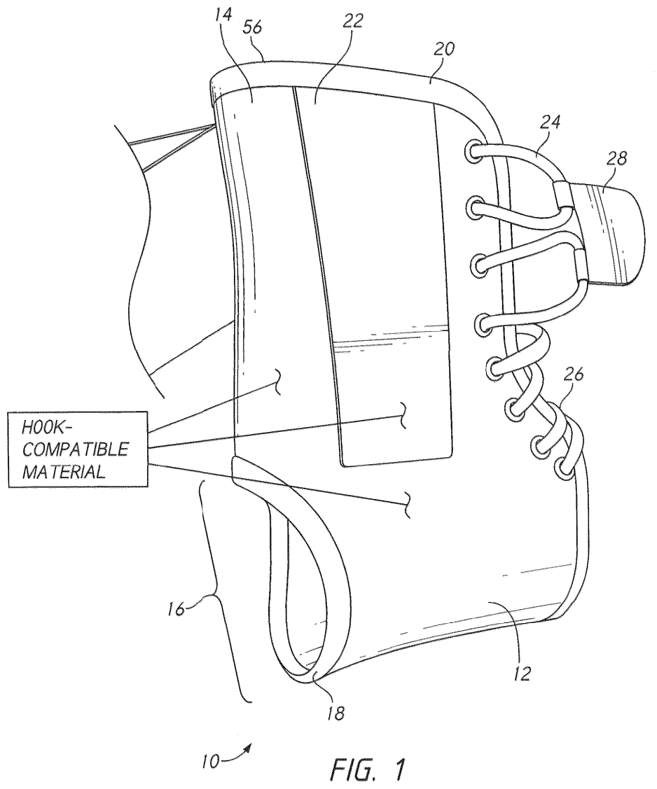

FIG. 1 provides a perspective view of the inventive ankle brace 10. The main body of the brace includes foot enclosure 12 joined to ankle enclosure 14. In the example shown, the foot enclosure and ankle enclosure are made as one unified piece. Soft materials are preferably used so that the brace can be worn directly over the skin or—alternatively—over a thin sock. In some versions the brace itself is made thin enough to wear inside a shoe.

The foot enclosure in the example of FIG. 1 is a large band passing around and under the foot. In other examples the foot enclosure may be less robust and may in fact be just a single broad strap or even a section of mesh netting.

Heel opening 16 allows the user's heel to protrude from the brace. An edge band is preferably sewn over all the edges to provide reinforcement. The heel opening includes edge band 18. Superior perimeter 56 includes edge band 20.

Hook-and-loop connections are used in many parts of the inventive brace. As those skilled in the art will know, hook-and-loop connections involve mating two hook-and-loop panels. As an example, a hook panel can be mated with a loop panel. In most instances the location of the hook panel and the loop panel may be reversed. The reader should also be aware that there are now many types of fabrics to which a hook panel will adhere. Many of these fabrics do not look like traditional deep-pile loop material, and instead just appear to be a smooth and soft fabric. Because these materials do not look like traditional loop material, they will be referred to as “hook-compatible material.” In the context of this application, the phrase “hook-compatible material” means any material to which a hook panel will adhere.

As noted in FIG. 1, the outward-facing surfaces of the foot enclosure and ankle enclosure are preferably covered with hook-compatible material. The inward-facing surfaces are preferably covered in a smooth and breathable material, since the inward-facing surfaces may be placed directly against the skin of the foot and ankle.

The brace shown in FIG. 1 may be worn on the right foot/ankle or the left foot/ankle without modification. Thus, it is impractical to refer to a lateral side and a medial side (since the definitions of these terms would vary with a right foot versus left foot application). Instead, the side facing the viewer in FIG. 1 is referred to as a first lateral side and the other side is referred to as a second lateral side.

In the invention some restraining straps are attached to the first and second lateral sides of the ankle enclosure. It is preferable to provide additional stiffness in the region where the attachments are made. First side reinforcement panel 22 is provided for this purpose. A second side reinforcement panel is provided on the second side of the ankle enclosure. This second side is not visible in FIG. 1.

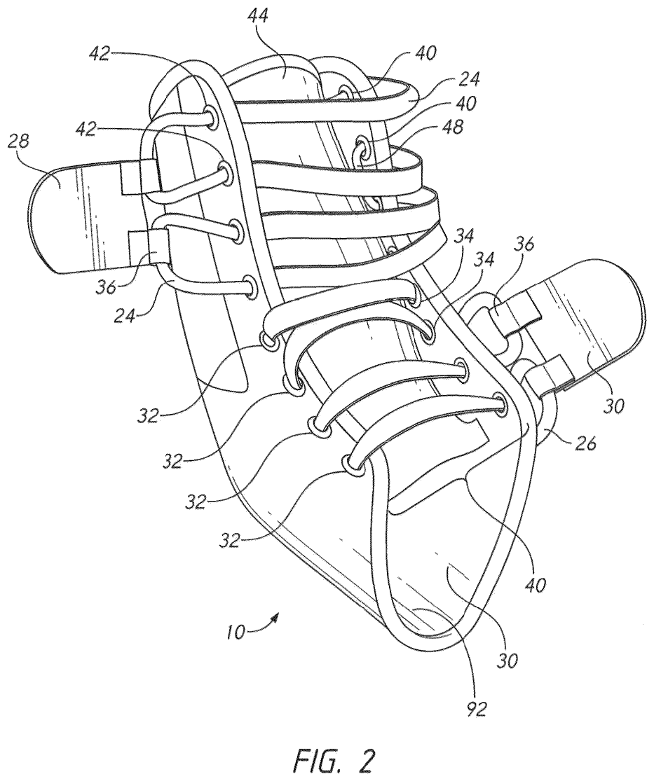

FIG. 2 provides a perspective view of the same embodiment as depicted in FIG. 1, but from a vantage point that is in front of the brace. The reader will note how the anterior portion of the ankle enclosure and the foot enclosure opens into adjustment break 46. Tongue 44 is secured across this adjustment break. The tongue is attached to the fabric on both sides of the break, in a manner consistent with a tongue of a boot.

The degree of closure of the adjustment break is controlled by a closure mechanism. In the example shown, the closure mechanism comprised two laces-upper lace 24 and lower lace 26. The tightness of upper lace 24 is controlled by pulling upper tab 28. The side of upper tab 28 facing the viewer in FIG. 2 includes a hook panel. Once the upper tab is pulled to a desired degree of tightness, the user moves the upper tab across the front of the brace and secures it to the hook-compatible surface on the brace's exterior.

The tightness of lower lace 26 is controlled by pulling lower tab 30. The side of lower tab 30 facing the viewer in FIG. 2 includes a hook panel. Once the lower tab is drawn taut, it can be moved across the front of the brace and secured to the hook-compatible surface on the brace's exterior.

Each of the tabs is preferably able to slide along the lace in order to allow the user to adjust an amount of taper produced when the lace is tightened. Upper tab 28—for example—is connected to upper lace 24 by a pair of loops 36. Each loop 36 is able to slide freely over upper lace 24. The two free ends of upper lace 24 are secured to ankle enclosure 14 (on the side of the adjustment break that is farthest from the user). All other portions of the upper lace pass freely through first side upper eyes 42, second side upper eyes 40, and the loops 36 on upper tab 28 (note exemplary internal loop 48 where the upper lace passes through). These features allow the user to evenly tighten the entire lace by pulling on upper tab 28. Further, the user can adjust the taper of adjustment break 46 by moving upper tab 28 up or down as it is tightened. “Adjusting the taper” refers to selectively making the top portion of the adjustment break larger than the bottom portion, or vice versa.

Lower tab 30 provides the same functionality for lower lace 26. The two free ends of the lower lace are secured to the brace (on the side closest to the user). All other portions of the lower lace slide freely through first side lower eyes 32 and second side lower eyes 34, and loops 36 on lower tab 30. These features allow the user to evenly tighten the lower lace by pulling on lower tab 30. The side of the lower tab facing the viewer in FIG. 2 includes a hook panel. Once the desired degree of tightness and taper are obtained for the lower lace, the user presses the lower tab against the hook-compatible exterior surfaces of the brace-thereby securing it in position.

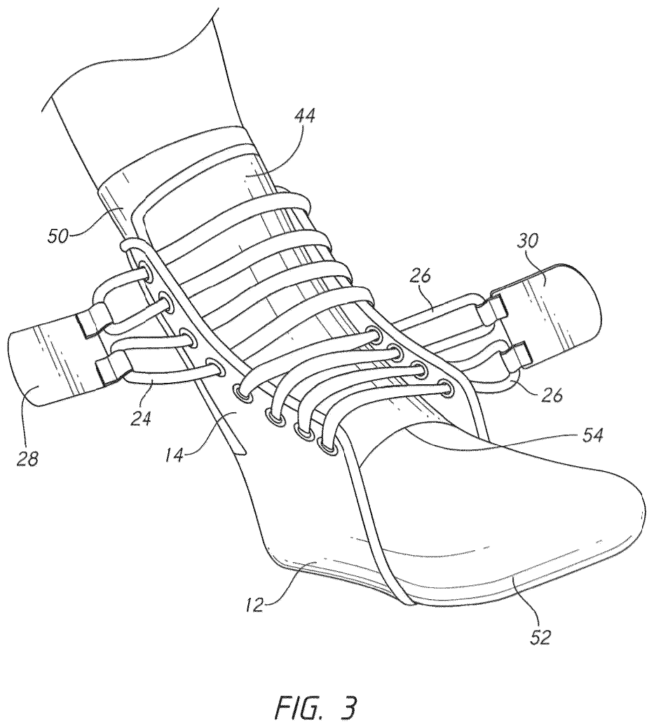

FIG. 3 shows a user's foot 52 with the inventive brace in position for installation. The brace is installed like a boot or sock. The user pushes the toes into the top of the brace behind tongue 44 and then pulls the brace up and onto the ankle. The forward portion of the foot extends well beyond inferior tongue edge 54 when installed. The upper portion of ankle enclosure 14 surrounds ankle 50. The user then adjusts lower tab 30 and upper tab 28 as desired.

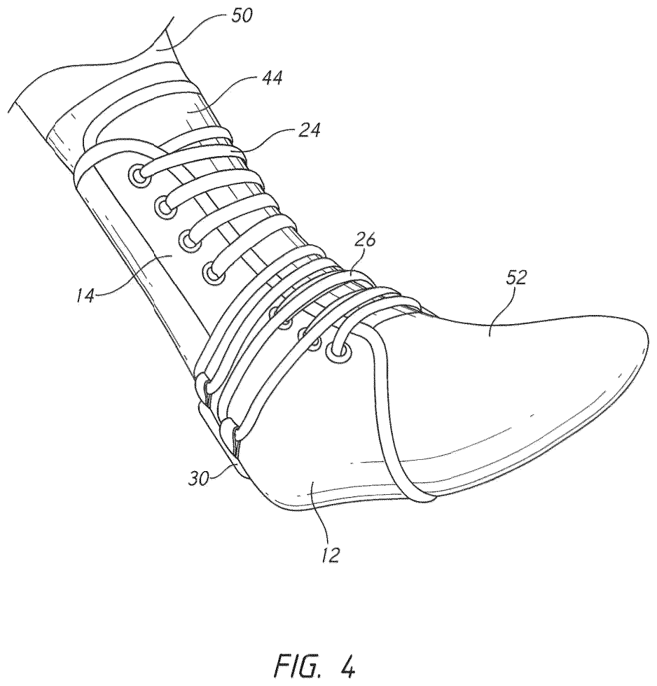

FIG. 4 shows the inventive brace after the upper and lower tabs have been secured. Ordinarily the lower tab 30 is tightened and secured first, but this is not always the case. The reader will note how lower tab 30 has been drawn around foot enclosure 12 and secured by pressing its hook panel against the hook-compatible outward-facing surface of the foot enclosure. The upper tab has been likewise drawn taut and secured. It is pressed against the far side of ankle enclosure 14 and is not visible in FIG. 4. The inventive brace-when thus installed-resembles a boot with the forward portion missing. The forward portion of foot 52 is free.

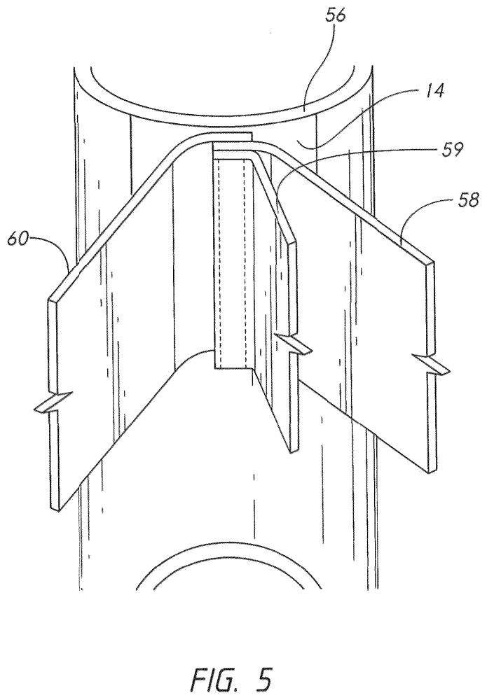

The rotation-limiting features of the inventive brace are provided by one or more additional straps. These additional straps are preferably attached to the upper rear portion of the ankle enclosure. FIG. 5 shows this portion of the brace-viewed from the back looking forward. Three straps are shown connected to ankle enclosure 14 proximate superior perimeter 56. These are: first side lateral strap 58, second side lateral strap 60, and superior securing strap 59. In the example shown the fixed end of these three straps is secured to the ankle enclosure via a double row of stitching. Each strap also has a distal end that is free.

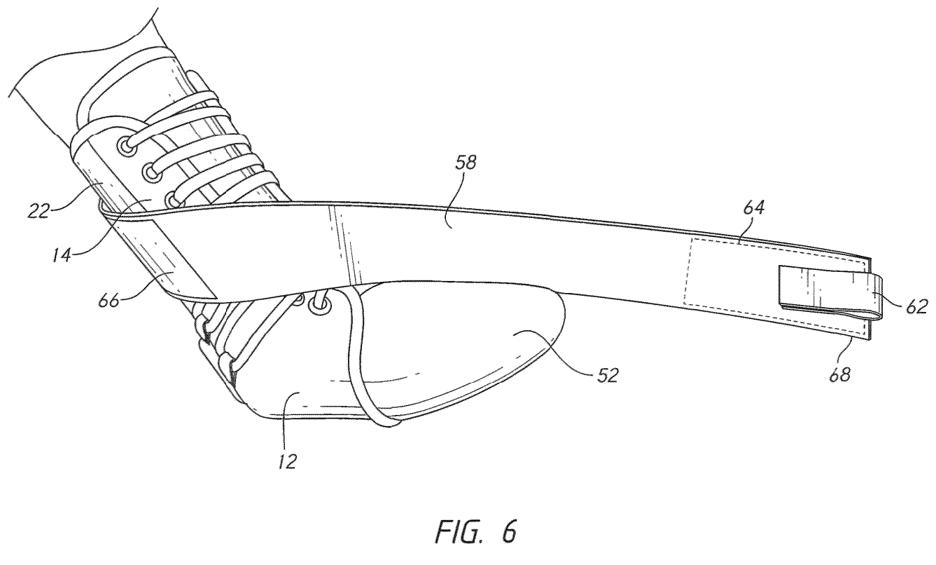

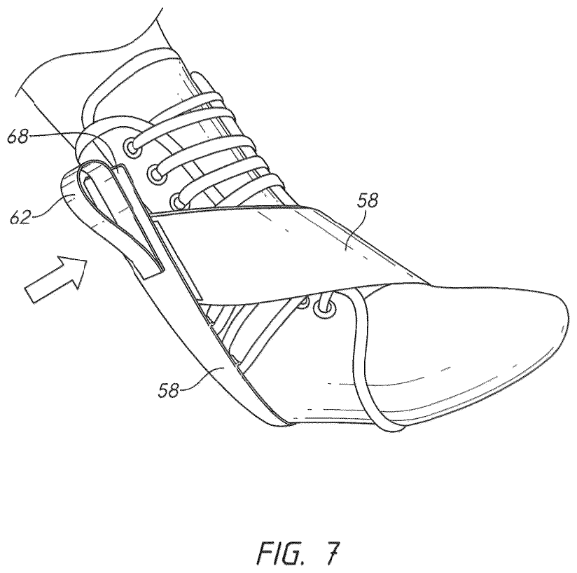

The installation of first side lateral strap 58 is shown in FIGS. 6 and 7. In FIG. 6, distal end 68 of first side lateral strap 58 is pulled around the first side of ankle enclosure 14 and over the anterior side of the ankle enclosure. The inward facing side of the strap 58 presses against ankle enclosure 14 and foot enclosure 12. The outward facing side of strap 58 includes hook-compatible panel 66. This panel is positioned to lie roughly in line with first side reinforcement panel 22 on the first side of the ankle enclosure.

In FIG. 6 the reader will observe that distal end 68 of first side lateral strap 58 includes pull tab 62. A user can hook a finger through pull tab 62 in order to apply greater tension to the strap. Hook panel 64 is located proximate the distal end, on the inward facing side of strap 58 (the side facing away from the viewer in FIG. 6). The operative concept of these features is for the user to maintain tension on strap 58 while pulling the strap around and under the sole of the foot. The strap is then pulled back up the first side of the brace.

FIG. 7 shows the completed securing of the strap. The user pulls upward on pull tab 62 until the desired tension is applied to first side lateral strap 58. The user then presses inward on distal end 68. Hook panel 64 (on the inward side of the strap, shown in FIG. 6), is pressed against the hook-compatible surface on first side reinforcement panel 22 and/or hook-compatible panel 66. Strap 58 is thereby secured in place.

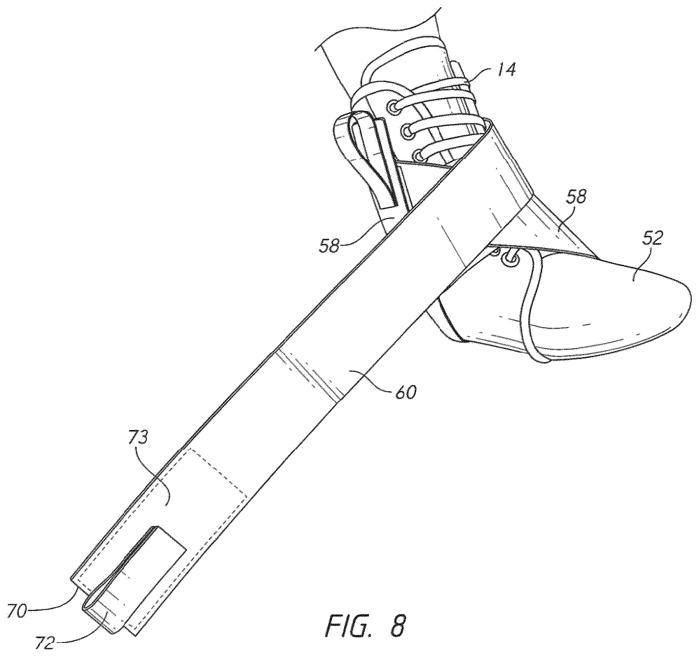

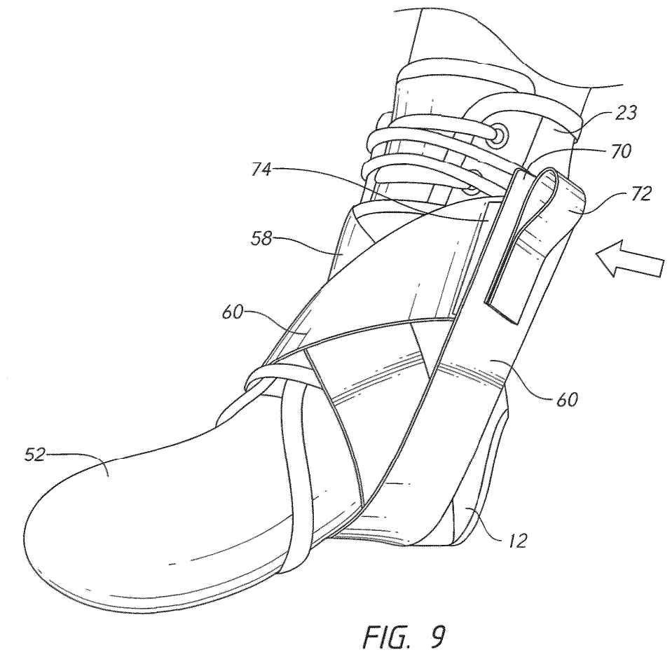

The operation of installing second side lateral strap 60 is essentially the mirror of the first side lateral strap. FIGS. 8 and 9 illustrate this process. Distal end 70 of second side lateral strap 60 is equipped with pull tab 72. A user employs this pull tab to pull strap 60 around the second side of ankle enclosure 14 and across the anterior portion of the ankle enclosure. In this example strap 58 is already installed so strap 60 is wrapped over strap 58 (The order of installation can be reversed if desired). Strap 60 includes hook panel 73 on its inward facing side. To complete the installation distal end 70 is passed under the sole of the foot and back up the second side of ankle enclosure 14.

FIG. 9 shows the installed configuration for second side lateral strap 60. Note that the outward facing side of strap 60 is supplied with a hook-compatible panel 74. This panel 74 is configured to roughly align with second side reinforcement panel 23 (which is also covered in hook-compatible material). Hook panel 73 (on the inward side of the strap 60, shown in FIG. 8) is pressed against the hook-compatible surface on second side reinforcement panel 23 and/or hook-compatible panel 74. Strap 60 is thereby secured in place.

The reader will thereby observe how the two lateral straps 58, 60 define a “FIG. 8” pattern over the ankle and foot. The intention of this configuration is to limit excess pronation (eversion) and/or excess supination (inversion). The “FIG. 8” pattern provides good protection, provided that the straps do not slip relative to each other (such as strap 60 slipping over strap 58). It is also important that the strap or straps does not slip relative to the sole region of foot enclosure 12. In looking at FIG. 9, the reader will readily appreciate that if the sole region of foot enclosure 12 easily slips along the inward facing surfaces of the straps 58, 60, then excess pronation and/or supination can still occur. The present invention includes additional features configured to limit unwanted slippage.

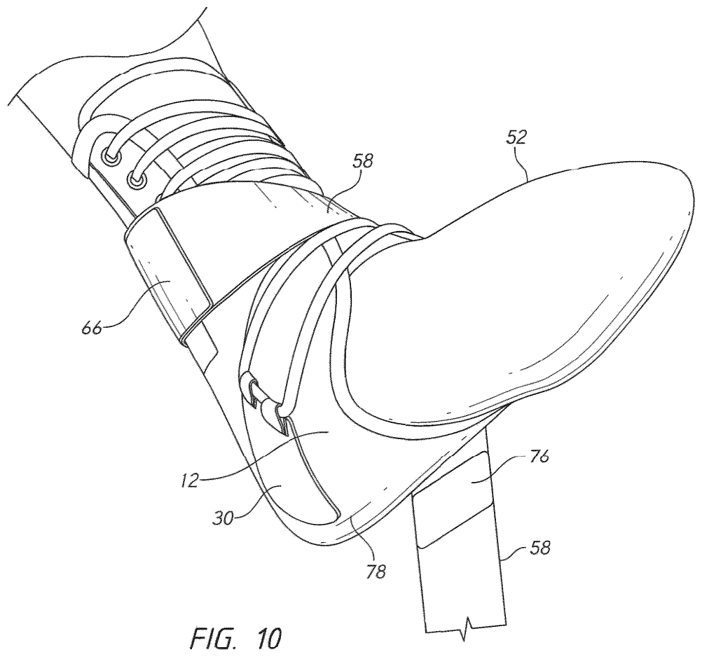

FIG. 10 shows the slippage limiting feature added to first side lateral strap 58. FIG. 10 corresponds to the configuration shown in FIG. 6, but with first side lateral strap 58 pulled further around the second side of the inventive brace. The outward facing surface of foot enclosure 12 is generally provided with a hook-compatible covering. Inferior region 78 in particular is provided with a hook-compatible covering. The inward facing surface of first side lateral strap 58 is provided with hook panel 76. Hook panel 76 is positioned so that it will engage the hook-compatible covering in inferior region 78 when strap 58 is pulled taut and secured. This engagement locks the portion of strap 58 passing under the sole to inferior region 78 of foot enclosure 12. As a result, the portion of strap 58 passing under the sole is not able to slip relative to inferior region 78.

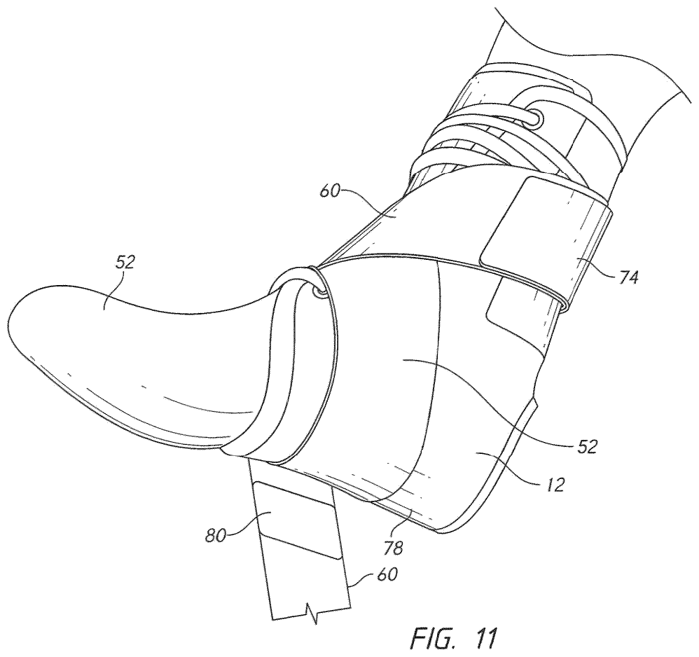

FIG. 11 shows the analogous feature for second side lateral strap 60. The inward facing surface of second side lateral strap 60 is provided with hook panel 80. Hook panel 80 is positioned so that it will engage the hook-compatible covering in inferior region 78 when strap 60 is pulled taut and secured. This engagement locks the portion of strap 60 passing under the sole to inferior region 78 of foot enclosure 12. As a result, the portion of strap 60 passing under the sole is not able to slip relative to inferior region 78.

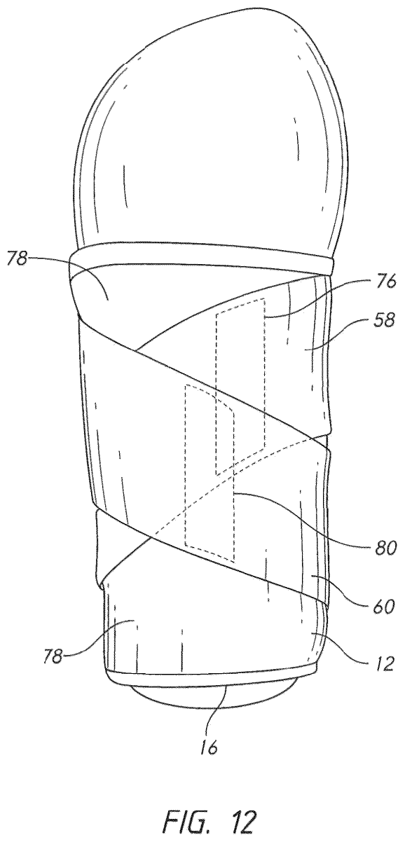

FIG. 12 shows a bottom view with first side lateral strap 58 and second side lateral strap 60 in the installed configuration. The reader will note how hook panel 76 is engaged to inferior region 78. Second side lateral strap 60 passes beneath first side lateral strap 58 (in this example). Much of hook panel 80 lies beneath first side lateral strap 58 and is therefore not engaged. However, a significant posterior portion of hook panel 80 directly contacts and engages inferior region 78. There is sufficient contact area to lock second side lateral strap 60 to inferior region 78. Thus, the portion of second side lateral strap 60 passing beneath the sole of the foot does not slip relative to foot enclosure 12.

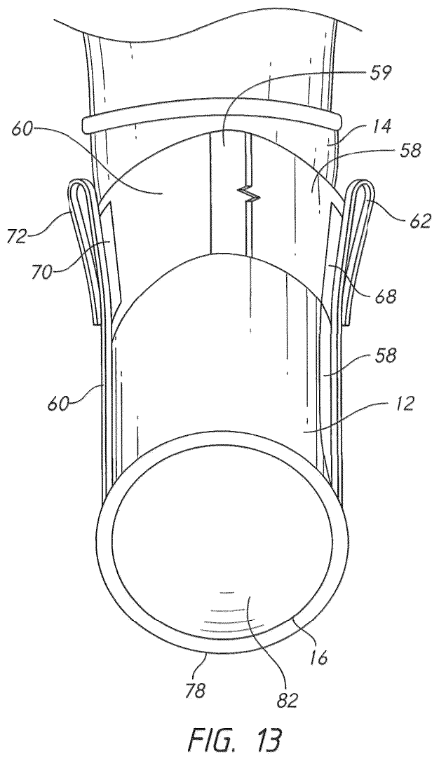

FIG. 13 shows the same assembly from the rear, looking forward. The reader will note how first side lateral strap 58 is secured to the first side of the ankle enclosure 14. The reader will also note how second side lateral strap 60 is secured to the second side of the ankle enclosure. The portion of each strap 58, 60 passing beneath the inferior region of foot enclosure 12 is locked to the foot enclosure via the engagement of hook panels 76, 80 (which are not visible in FIG. 13 but are shown in FIG. 12).

In looking at FIG. 13, the reader will understand that if the foot starts to rotate clockwise with respect to ankle enclosure 14, then the portion of first side lateral strap 58 spanning the length from distal end 68 to hook panel 76 will draw taut and prevent further clockwise rotation. If, on the other hand, the foot starts to rotate counterclockwise with respect to ankle enclosure 14 then the portion of second side lateral strap 60 spanning the length from distal end 70 to hook panel 80 will draw taut and prevent further rotation.

The wearer of the brace—or an experienced medical provider—is able to finely tune the amount of inversion or eversion the inventive brace permits. From the vantage point of FIG. 13, the user may be primarily concerned with preventing any significant clockwise rotation of the foot relative to ankle enclosure 14. The user adjusts permissible rotation by setting the position of first side lateral strap 58. First the user wraps first side lateral strap around as shown in FIG. 10. Next the user engages hook panel 76 to inferior region 78. Then-again looking at FIG. 13—the user grasps pull tab 62 and pulls upward on distal end 68 until the desired position of the lower foot is set. Then—while maintaining tension on strap 58—the user presses the hook panel on the inward facing side of distal end 68 laterally against the hook-compatible surfaces on the ankle enclosure and on the proximal portion of strap 58 itself (hook compatible panel 66 shown in FIG. 6). Straps 58, 60 are preferably made of inelastic material so that the positon set by the user is maintained firmly.

The user can set a desired position for counterclockwise rotation of the foot by setting the desired position of second side lateral strap 60. The user can set a relatively fixed rotational position for the foot (pronation/supination) and lock the foot in that position by placing significant tension on both straps 58, 60. The engagement of the hook panels 76, 80 prevents unwanted slippage and allows the setting of tension on the straps to create and maintain a desired orientation between the foot and the ankle.

Turning back to FIG. 5, the reader will recall the presence of superior securing strap 59. This strap has a proximal end attached to the upper rear portion of ankle enclosure 14 as shown and a free distal end. The securing strap is configured to wrap around the upper portion of the brace and thereby secure and guard the distal end of the lateral straps and other components. The superior securing strap has a hook panel on its inward facing side proximate its distal end. The superior strap's outward facing surface is covered in hook-compatible material.

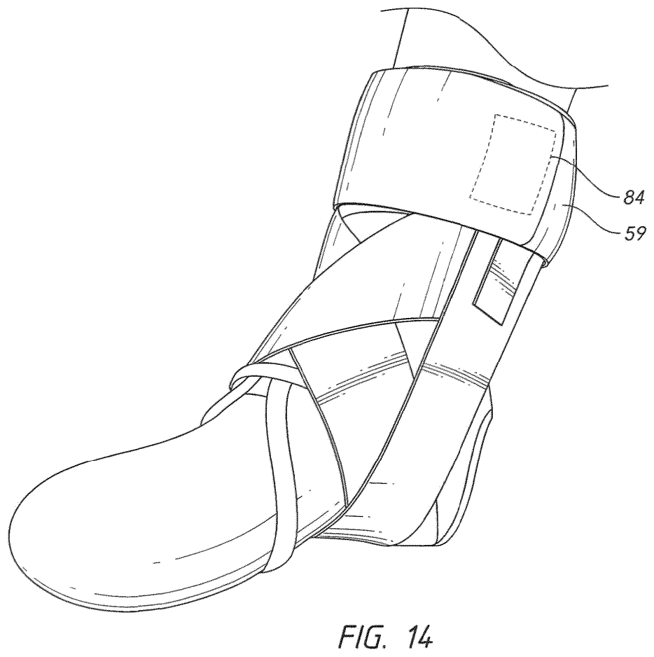

This combination of features allows the superior securing strap to be looped around the upper portion of the inventive brace and then secured to itself. FIG. 14 shows superior securing strap 59 after it has been looped around the upper portion of the brace. Hook panel 84 (on the inward facing surface of the distal end of the superior securing strap) has been secured to the outward facing surface of the strap. The reader will note how the superior securing strap covers the hook tabs and distal end of the two side lateral straps 58, 60. The superior securing strap thereby prevents unwanted detachment or movement of the two side lateral straps.

Although the preceding descriptions present considerable detail they should be properly viewed as illustrating preferred embodiments of the present invention rather than limiting the scope of the invention. Many more embodiments following the same principles will occur to those skilled in the art. As one example, some embodiments will include only a single lateral strap instead of a pair of opposing lateral straps. Accordingly, the scope of the invention should be fixed by the following claims rather than by the examples given.

Having described my invention, I claim:

1. A method for reinforcing a human ankle and foot while limiting inversion and eversion of said ankle, comprising:
(a) providing an ankle brace, including,
(i) a foot enclosure having a superior region and an inferior region, with said inferior region including a hook-compatible area,
(ii) an ankle enclosure connected to said foot enclosure, said ankle enclosure having a first lateral side, a second lateral side, an anterior side, and a posterior side,
(iii) said first lateral side and said second lateral side of said ankle enclosure having hook-compatible material,
(iv) a first side lateral strap having a first fixed end attached to said ankle enclosure and a first distal end,
(v) said first side lateral strap having a first inward facing side and a first outward facing side,
(vi) a second side lateral strap having a second fixed end attached to said ankle enclosure and a second distal end,
(vii) said second side lateral strap having a second inward facing side and a second outward facing side,
(viii) said first side lateral strap having a first hook-compatible panel on said first outward facing side, a first hook panel on said first inward facing side, and a second hook panel on said first inward facing side proximate said first distal end,
(ix) said second side lateral strap having a second hook-compatible panel on said second outward facing side, a third hook panel on said second inward facing side, and a fourth hook panel on said second inward facing side proximate said second distal end,
(x) said first side lateral strap being inelastic from said first hook panel to said second hook panel,
(xi) said second side lateral strap being inelastic from said third hook panel to said fourth hook panel;
(b) securing said foot enclosure to said human foot;
(c) securing said ankle enclosure to said human ankle;
(d) wrapping said first side lateral strap around said anterior portion of said ankle enclosure from said first lateral side toward said second lateral side and then under said inferior region of said foot enclosure;
(e) connecting said first hook panel on said first side lateral strap to said hook-compatible area on said inferior region of said foot enclosure;
(f) setting an inversion limit for said human ankle by pulling on said first distal end of said first side lateral strap in order to place said first distal end in a desired location and then engaging said second hook panel on said first side lateral strap to said first hook-compatible panel and said hook-compatible material on said first lateral side of said ankle enclosure;
(g) wrapping said second side lateral strap around said anterior portion of said ankle enclosure from said second lateral side toward said first lateral side and then under said inferior region of said foot enclosure
(h) connecting said third hook panel on said second side lateral strap to said hook-compatible area on said inferior region of said foot enclosure; and
(i) setting an eversion limit for said human ankle by pulling on said second distal end of said second lateral strap in order to place said second distal end in a desired location and then engaging said fourth hook panel on said second side lateral strap to said second hook-compatible panel and said hook-compatible material on said second lateral side of said ankle enclosure.

2. The method for reinforcing a human ankle and foot as recited in claim 1, wherein said ankle brace further comprises:
(a) providing a first pull tab proximate said first distal end of said first side lateral strap; and
(b) providing a second pull tab proximate said second distal end of said second side lateral strap.

3. The method for reinforcing a human ankle and foot as recited in claim 1, wherein said ankle brace further comprises:
(a) an anterior break in said ankle enclosure; and
(b) said anterior break being adjustably closable by an upper lace assembly.

4. The method for reinforcing a human ankle and foot as recited in claim 1, wherein said ankle brace further comprises:
(a) a superior break in said foot enclosure; and
(b) said superior break being adjustably closable by a lower lace assembly.

5. The method for reinforcing a human ankle and foot as recited in claim 3, wherein said ankle brace further comprises an upper pull tab for adjusting a tension of said upper lace assembly.

6. The method for reinforcing a human ankle and foot as recited in claim 4, wherein said ankle brace further comprises a lower pull tab for adjusting a tension of said lower lace assembly.

7. The method for reinforcing a human ankle and foot as recited in claim 6, wherein:
(a) said lower pull tab has a hook panel on an inward facing side; and
(b) said foot enclosure includes a hook-compatible covering configured to engage said hook panel on said lower pull tab.

8. A method for reinforcing a human ankle and foot while limiting inversion and eversion of said ankle, comprising:
(a) providing an ankle brace, including,
(i) a foot enclosure having a superior region and an inferior region, with said inferior region including a hook-compatible area,
(ii) an ankle enclosure connected to said foot enclosure, said ankle enclosure having a first lateral side, a second lateral side, an anterior side, and a posterior side,
(iii) said first lateral side and said second lateral side of said ankle enclosure having hook-compatible material,
(iv) a first side lateral strap having a first fixed end attached to said ankle enclosure and a first distal end,
(v) said first side lateral strap having a first inward facing side and a first outward facing side,
(vi) a second side lateral strap having a second fixed end attached to said ankle enclosure and a second distal end,
(vii) said second side lateral strap having a second inward facing side and a second outward facing side,
(viii) said first side lateral strap having a first hook panel on said first inward facing side, and a second hook panel on said first inward facing side proximate said first distal end,
(ix) said second side lateral strap having a third hook panel on said second inward facing side, and a fourth hook panel on said second inward facing side proximate said second distal end,
(x) said first side lateral strap being inelastic from said first hook panel to said second hook panel,
(xi) said second side lateral strap being inelastic from said third hook panel to said fourth hook panel;
(b) securing said foot enclosure to said human foot;
(c) securing said ankle enclosure to said human ankle;
(d) wrapping said first side lateral strap around said anterior portion of said ankle enclosure from said first lateral side toward said second lateral side and then under said inferior region of said foot enclosure;
(e) connecting said first hook panel on said first side lateral strap to said hook-compatible area on said inferior region of said foot enclosure;
(f) setting an inversion limit for said human ankle by pulling on said first distal end of said first side lateral strap in order to place said first distal end in a desired location and then engaging said second hook panel on said first side lateral strap to said hook-compatible material on said first lateral side of said ankle enclosure;
(g) wrapping said second side lateral strap around said anterior portion of said ankle enclosure from said second lateral side toward said first lateral side and then under said inferior region of said foot enclosure
(h) connecting said third hook panel on said second side lateral strap to said hook-compatible area on said inferior region of said foot enclosure; and
(i) setting an eversion limit for said human ankle by pulling on said second distal end of said second lateral strap in order to place said second distal end in a desired location and then engaging said fourth hook panel on said second side lateral strap to said hook-compatible material on said second lateral side of said ankle enclosure.

9. The method for reinforcing a human ankle and foot as recited in claim 8, wherein said ankle brace further comprises:
(a) providing a first pull tab proximate said first distal end of said first side lateral strap; and
(b) providing a second pull tab proximate said second distal end of said second side lateral strap.

10. The method for reinforcing a human ankle and foot as recited in claim 8, wherein said ankle brace further comprises:
(a) an anterior break in said ankle enclosure; and
(b) said anterior break being adjustably closable by an upper lace assembly.

11. The method for reinforcing a human ankle and foot as recited in claim 8, wherein said ankle brace further comprises:
(a) a superior break in said foot enclosure; and
(b) said superior break being adjustably closable by a lower lace assembly.

12. The method for reinforcing a human ankle and foot as recited in claim 10, wherein said ankle brace further comprises an upper pull tab for adjusting a tension of said upper lace assembly.

13. The method for reinforcing a human ankle and foot as recited in claim 11, wherein said ankle brace further comprises a lower pull tab for adjusting a tension of said lower lace assembly.

14. The method for reinforcing a human ankle and foot as recited in claim 13, wherein:
(a) said lower pull tab has a hook panel on an inward facing side; and
(b) said foot enclosure includes a hook-compatible covering configured to engage said hook panel on said lower pull tab.

15. A method for reinforcing a human ankle and foot while limiting inversion and eversion of said ankle, comprising:
(a) providing an ankle brace, including,
(i) a foot enclosure having a superior region and an inferior region,
(ii) an ankle enclosure connected to said foot enclosure, said ankle enclosure having a first lateral side, a second lateral side, an anterior side, and a posterior side,
(iii) a first side lateral strap having a first fixed end attached to said ankle enclosure, a first middle portion, and a first distal end,
(iv) said first side lateral strap having a first inward facing side and a first outward facing side,
(v) a second side lateral strap having a second fixed end attached to said ankle enclosure, a second middle portion, and a second distal end,
(vi) said second side lateral strap having a second inward facing side and a second outward facing side,
(vii) said first side lateral strap being inelastic from said first middle portion to said first distal portion,
(viii) said second side lateral strap being inelastic from said second middle portion to said second distal portion;
(b) securing said foot enclosure to said human foot;
(c) securing said ankle enclosure to said human ankle;
(d) wrapping said first side lateral strap around said anterior portion of said ankle enclosure from said first lateral side toward said second lateral side and then under said inferior region of said foot enclosure;
(e) connecting said first middle portion of said first side lateral strap to said inferior region of said foot enclosure;
(f) setting an inversion limit for said human ankle by pulling on said first distal end of said first side lateral strap in order to place said first distal end in a desired location and then connecting said first distal end to said first lateral side of said ankle enclosure;
(g) wrapping said second side lateral strap around said anterior portion of said ankle enclosure from said second lateral side toward said first lateral side and then under said inferior region of said foot enclosure;
(h) connecting said second middle portion of said second side lateral strap to said inferior region of said foot enclosure; and
(i) setting an eversion limit for said human ankle by pulling on said second distal end of said second lateral strap in order to place said second distal end in a desired location and then connecting said second distal end to said second lateral side of said ankle enclosure.

16. The method for reinforcing a human ankle and foot as recited in claim 15, wherein said ankle brace further comprises:
(a) providing a first pull tab proximate said first distal end of said first side lateral strap; and
(b) providing a second pull tab proximate said second distal end of said second side lateral strap.

17. The method for reinforcing a human ankle and foot as recited in claim 15, wherein said ankle brace further comprises:
(a) an anterior break in said ankle enclosure; and
(b) said anterior break being adjustably closable by an upper lace assembly.

18. The method for reinforcing a human ankle and foot as recited in claim 15, wherein said ankle brace further comprises:
(a) a superior break in said foot enclosure; and
(b) said superior break being adjustably closable by a lower lace assembly.

19. The method for reinforcing a human ankle and foot as recited in claim 17, wherein said ankle brace further comprises an upper pull tab for adjusting a tension of said upper lace assembly.

20. The method for reinforcing a human ankle and foot as recited in claim 18, wherein said ankle brace further comprises a lower pull tab for adjusting a tension of said lower lace assembly.
